(19) United States
(12) Patent Application Publication
(10) Pub. No.: US 2008/0183094 A1
(43) Pub. Date: Jul. 31, 2008

(54) Method and Device For Recovering and Analyzing Respiratory Condensates

(76) Inventors: Dirk Schonfuss, Berlin (DE); Jan Szeponik, Berlin (DE); Ingrid Gall, Zepernick (DE)

Correspondence Address: THELEN REID BROWN RAYSMAN & STEINER LLP, P.O. BOX 640640, SAN JOSE, CA, 95164-0640, US

(21) Appl. No.: 11/912,391

(22) PCT Filed: Apr. 25, 2006
(86) PCT No.: PCT/EP2006/004227

§ 371 Date: Nov. 6, 2007

(30) Foreign Application Priority Data
Apr. 25, 2005 (DE) 10 2005 020 102.4

(51) Int. Cl. A61B 5/08

(52) U.S. Cl. 600/532

(57) ABSTRACT

The invention relates to a method for analyzing components of exhaled air with the aid of electrochemical sensors and/or biosensors. Said components are determined by measuring and evaluating signals generated by one or several of the sensors during contact with said substances or reaction products obtained from said substances. The substances that are to be analyzed are also condensed when the water contained in breathing gas is condensed on a condensation surface. The obtained respiratory condensate is fed directly to the sensors while the condensation surface is functionalized or activated such that an interaction or a chemical reaction takes place with the condensate and/or the substances that are to be analyzed during the condensation process, whereby the signal generated on the sensors is influenced. The invention further relates to a device for recovering respiratory condensates. Said device comprises at least one sensor unit which is placed on a basic element (a fixture or a support) and is provided with an appropriately functionalized or activated condensation surface in addition to the sensors. The inventive device also comprises at least one Peltier element and a heat-conducting bridge. In a preferred embodiment, the basic element can be connected to a head fixture and can thus be positioned directly in the respiratory flow directly in front of the mouth of the person wearing the device and be connected to a portable power supply system and an electronic evaluation system as required.

The invention relates to a method for analyzing constituents of exhaled air using electrochemical sensors and/or biosensors. The determination is performed by measuring and analyzing signals generated by one or more of the sensors on contact with these substances or with reaction products formed from these substances. These substances to be analyzed are also condensed in condensation of the water contained in the respiratory gas on a condensation surface. The resulting respiratory condensate is sent directly to the sensors. The condensation surface is functionalized and/or activated in such a way as to yield an interaction, i.e., a chemical reaction with the condensate and/or the substances to be analyzed during the condensation process, so thereby influencing the signal generated by the sensors. The invention also relates to a device for collecting respiratory condensates, comprising at least one sensor unit on a base element (a holder and/or carrier), said sensor unit having a suitably functionalized or activated condensation surface in addition to the sensors. This device also comprises at least one Peltier element and a heat conducting bridge. In a preferred embodiment of the invention, the base element may be connected to a headset and thereby positioned directly in front of the wearer's mouth, directly in the respiratory stream, optionally connected to a portable power supply and electronic analyzer system.

There are several known variants and devices for collecting respiratory condensate and determining the concentration of substances in the respiratory condensate. DE 199 51 204 C2 describes a method for analyzing the constituents of exhaled air, in which the exhaled air is cooled until its aerosol and vapor constituents are condensed to obtain a predetermined amount of sample. The predetermined amount of sample is determined by completely filling or saturating a storage unit or filter. The substances present in the condensate are then determined on one or more electrochemical sensors downstream from the storage layer or filter, such that the respiratory condensate diffuses through the filter or the storage layer to the sensors. One disadvantage of this and similar approaches is that the respiratory condensate is first collected in the filter or the storage layer and thus a large portion of the sample is withheld from the analytical process and short-lived substances (e.g., radicals) may be present only partially or may no longer be present at all because only after complete filling of the filter or the storage layer with respiratory condensate does the latter reach the downstream sensors through diffusion. The disadvantages include the risk of infection and contamination of the sample as well as the disinfection required after each analysis. DE 101 375 65 A1 describes the determination of parameters of the respiratory condensate with the help of a closed cassette in which the sensors themselves as well as buffer solutions, calibration solutions and possibly also dilution solutions are accommodated in storage containers or in replaceable cartridges to alter the conductivity of the sample. The solutions are dispensed from the storage containers to the sensors for the purpose of measurement, sensor conditioning or sensor calibration with the help of a special apparatus which acts on the storage container and is not part of the cassette. The sample solution, the respiratory condensate, is drawn or injected into the cassette out of a sample container or a sample collecting system before the measurement and, before being dispensed to the sensors, is subjected to mixing with substances or solutions that are accommodated inside or outside the cassette for the purpose of dilution and/or changing the ion concentration and/or conductivity. After the measurement, the cassette is discarded. One disadvantage here is the great complexity in terms of technical equipment for collecting an adequate amount of sample, which is done with a separate apparatus, after which the sample must still be subjected to dilution, if necessary. Another disadvantage is the great complexity in terms of technical equipment required for operation of the cassette for pretreatment and analysis of the sample as well as the high cost of manufacturing and cost of materials for the cassette itself. These disadvantages must be seen against the advantages of prompt and continuous diagnostic tests that are mobile in the sense that the test subject can carry the analyzer device with him for an unlimited period of time.

Another possibility for determining organic substances present in the gas phase is described in EP 0 634 488 A2, which discloses a biosensor comprising a thick film substrate printed with electrodes to which a moisture absorbing layer is applied, said layer containing an enzyme that reacts with the substance to be determined in the gas phase. Use of this biosensor for the analysis has the disadvantage that introducing the sensor into the gas phase to be analyzed determines the starting point of sample collection by absorption, and its rate depends on, first, the starting condition of the sensor and, second, the environment in the gas (e.g., moisture and oncoming flow conditions on the sensor surface). It is therefore necessary to establish appropriately complex control and/or monitoring of ambient conditions to ensure a defined start of the measurement and thereby prevent incorrect measurements due to artifacts during the as yet incomplete absorption of the sample from the gas phase. This method is not suitable for determining the concentration of substances in a relatively heterogeneous gas flow, such as that which occurs in exhalation.

The object of the present invention is therefore to provide a method for determining substances in the exhaled air based on respiratory condensates and using sensors such that the method reliably yields actual measured values. The object of the present invention was also to develop an inexpensive device that would not cause a burden on the test subject and may optionally also be used as a mobile device, allowing actual analysis of the substances and guaranteeing the most prompt possible and continuous diagnostic testing and monitoring of diseases and physiological processes of the respiratory organs on the basis of determination of the concentration of substances which are present in the respiratory condensate and are relevant for these processes.

The object of the present invention is achieved with the characterizing features of process claim 1 and device claim 10. Advantageous embodiments of the method and device are the subject matter of the subclaims.

Electrochemical sensors and/or biosensors are used in combination with a power supply system and an electronic measurement, control and analysis system for the inventive method for determination of substances in the exhaled air on the basis of respiratory condensates. The respiratory air condensates are deposited on a functionalized condensation surface which is cooled and connected to the sensors. According to this invention, the condensation surface is functionalized with substances that enter into an interaction or chemical reaction with substances in the condensate of the exhaled air and/or which alter the composition of the condensate. The droplets of condensate formed due to cooling reach the sensors through gravitational force, capillary forces and/or surface effects, depending on the geometric dimensions of the condensation surface, whereby the signals generated by the substances and/or the reactions can be determined by means of measurement, control and analysis systems. Condensation surfaces that are coated with a functionalized or activated organic polymer or mixtures thereof are especially preferred. These are preferably layers of polyvinyl, polystyrene, polyacrylate, polyurethane or cellulose derivatives and/or silicon polymer compounds. Functionalized or activated condensation surfaces in the sense of the present invention mean that they have polymers with substances which enter into an interaction and/or trigger a chemical reaction with substances in the exhaled air. For example, the polymer may contain inorganic salts, e.g., the chlorides KCl or NaCl, which dissolve in the respiratory condensate and therefore lead to a change (increase) in conductivity and allow measurements with electrochemical sensors. Polymer layers of polyurethane or polyurethane mixtures, cellulose derivatives and/or silicon polymer compounds are preferably used.

Suitably functionalized or activated organic polymers are produced, e.g., by adding salts or other additives to polymer suspensions or mixtures of polymer suspensions before crosslinking the polymer. The salts or other additives become incorporated into the polymer layer due to the crosslinking of the polymer. Another possibility of functionalizing polymers is to immobilize chemical reactants, e.g., enzymes or substances to be detected in the polymers or at the surfaces thereof. In addition, by using certain polymers and by chemical or physical surface treatment thereof, the wetting properties of these polymers can be influenced; this has effects on the droplet size, the cohesion of droplets of condensate and thus on the entire course of sample collection.

The surface of the condensation surfaces is designed so that the resulting droplets of condensates coalescence to form larger and larger droplets. Within this relatively short period of time, they surprisingly absorb up the substances that are present in the polymer, for example. This makes it possible to influence the conductivity of the condensate, for example, as already mentioned. Furthermore, the substances to be determined may react with the enzymes immobilized in the polymer layer, for example, to form easily detectable reactants. For example, lactate present in respiratory condensate may react in a reaction catalyzed by lactate oxidase to yield pyruvate and hydrogen peroxide, which can easily be detected electrochemically. However, another possibility is functionalization of the condensation surface by covalent bonding of substances to the condensation surface, where these substances enter into an interaction or a chemical reaction with substances in the condensate of the exhaled air. For example, enzymes are bound directly to functionalized polymeric surfaces with the help of glutardialdehyde or to gold surfaces via thiols or to glass or ceramics via organosilicon compounds.

The inventive device for collecting respiratory condensates and immediately determining substances present in the respiratory air includes at least one sensor unit, at least one Peltier element for cooling and at least one heat conducting bridge between the Peltier element and the condensation surface for cooling, arranged on a base element in the form of a carrier or a holder with the proper contacts. The sensor unit has at least functionalized or activated condensation surface. In addition, the sensor unit comprises at least one sensor, with the condensation surface(s) and sensor(s) being joined in such a way that the condensate can reach the sensor directly. Electrochemical sensors and/or biosensors, which can be connected to power supply systems and electronic measurement, control and analysis systems are used. The device has the great advantage that in addition to normal standard devices in the clinical and laboratory area, portable systems suitable for point-of-care use may also be used.

After a certain period of time, which depends on the temperature of the cooled functionalized surface, its surface properties and its geometric dimensions, the collected condensate goes directly to the sensors, which are in the immediate vicinity of the cooled surface; the constituents present in the respiratory condensate can be determined with these sensors in combination with the corresponding electronic measurement and analysis equipment. Parameters of the respiratory condensate can be determined with the corresponding sensors, e.g., the hydrogen peroxide concentration, the lactate concentration or the ammonium concentration.

In a preferred variant, the temperature of the cooled surface and thus the point in time as well as the duration of collection of condensate can be controlled easily with the help of the Peltier element in combination with temperature sensors, which measure the ambient temperature and temperature of the cooled surface.

In another preferred embodiment, by subdividing the condensation surface into multiple condensation surfaces and their corresponding thermal insulation in combination with one or more Peltier elements, it is possible to control cascades or condensation sequences. A fluidics can be established through the design of the condensation surfaces with functionalized layers; interaction effects or chemical processes can be triggered in a argeted manner. The latter is accomplished, for example, by the fact that the condensate absorbs the substances which have been introduced in a targeted manner.

The sensor unit of the device for collecting and analyzing respiratory condensates has a functionalized condensation surface, which is constructed on the basis of polymer, metal or ceramic materials or a combination of these materials, and a surface on which the sensors are situated. The surfaces holding the sensors are preferably made of ceramic or plastic, especially thermoplastics. The condensation surface or surfaces are combined spatially with the elements carrying one or more sensors to form the sensor unit so that after a certain quantity of condensate has formed, it goes directly to the individual sensor or sensors, e.g., through gravitational force, capillary forces and/or surface effects, and the components of the respiratory condensate are determined. The shape and size of the condensation layer are determined and limited by a cover. This cover may be designed so that an additional wall runs at a slight distance above the surface of the sensor or sensors, thereby forming the space above the surface of the sensor or sensors which can be filled by means of capillary forces. The sensor unit is arranged on a base element, which may be a carrier or a holder into which the sensor unit may be inserted. The base element has corresponding contact paths, preferably terminal contacts for the sensors and for the transfer of heat from at least one Peltier element, which is also accommodated in this base element and may be connected to the measurement, analysis and power supply unit.

The inventive device has the advantage that, in addition to gravitational force, capillary forces and/or surface effects may also be employed in the design of the inventive device to induce and/or support the conveyance of condensate to the sensors or, on the other hand, to define the sample volume applied to the sensors. Due to the terminal contacts of the devices that are preferably selected, this ensures that the sensor unit can be replaced rapidly and in an uncomplicated manner. It is therefore possible to use various configurations of such sensor units having different sensors and polymer coatings for different applications. In a preferred embodiment of the inventive device, a holder with a sensor unit and a Peltier element is accommodated in a holder (headset) which can be worn by a test subject to thereby ensure a reasonable method of prompt and continuous collection of condensate. Individual sensors or combinations of disposable or reusable sensors based on electrochemical or optical principles or biosensors may be used as the sensors, depending on the requirements of the parameters and substances to be determined.

### Exemplary Embodiments

### EXAMPLE

### Determination of Lactate

A ceramic substrate that has been provided with a two-electrode structure in the thick-layer technique also has a condensation surface which is covered with a hydroxyethyl cellulose layer functionalized with the lactate oxidase enzyme. The condensation surface and the two-electrode structure on the ceramic together form the sensor unit which is inserted into a holder equipped with a Peltier element, a heat conducting bridge and electric spring contacts and is connected to a measurement, control and regulating unit. A voltage of +450 mV is applied between the two electrodes of the two-electrode structure; the hydrogen peroxide formed as a byproduct in the reaction with lactate present in the respiratory condensate, catalyzed by the lactate oxidase enzyme, is oxidized electrochemically at this voltage. The condensation surface is first cooled from room temperature to 14° C. Then the test subject directs his breath at the condensation surface of the sensor unit until a certain amount of condensate has collected and reached the electrode structure. Wetting of the sensor structure with the respiratory condensate is manifested in a rapid increase and decrease in the sensor current, depending on the lactate concentration. Thirty seconds after wetting of the electrode structure, an electric current of 12 nA was measured for a lactate content of 50 μmol/L in the respiratory condensate, whereas an electric current of 5 nA was measured with a lactate-free comparative sample, also 30 seconds after wetting (see FIG. 5).

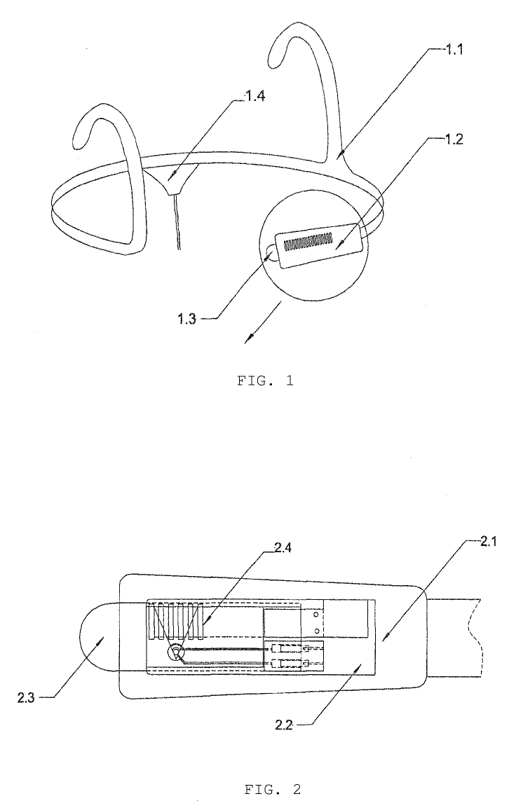

In addition, FIGS. 1 through 4 show preferred embodiments of the device, although the invention is not limited to these embodiments.

FIG. 1 shows a schematic diagram of a headset (1.1) with an inventive device to be positioned in front of a test subject's mouth, said device being in the form of a headset (1.2) with an inserted sensor unit (1.3), a Peltier element and a heat conducting bridge. The sensor contacts and the Peltier element and/or multiple Peltier elements contained in the headset may be connected by suitable lines (1.4) to a measurement control and power supply unit (not shown here) which can worn on the body.

FIG. 2 shows a headset (2.2) which is surrounded by a housing (2.1) that is provided with slots (2.4) for heat exchange and mass exchange with the environment, with a sensor unit (2.3) inserted into this holder, a Peltier element and a heat conducting bridge.

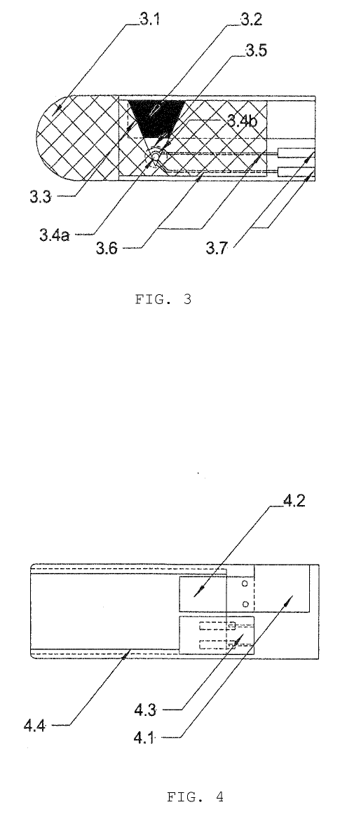

FIG. 3 shows schematically a sensor unit with a handle piece (3.1), a cover (see hatching) which is provided with a cutout (3.3) that exposes a uniform condensation surface (3.2) and the reaction surface of an individual biosensor structure (3.5) which is shown here schematically. The biosensor consists of electrode structures, for example, namely the working electrode (3.4a), a counterelectrode/reference electrode (3.4b), discharge structures (3.6) and contact structures (3.7) printed on a thermally insulating layer. The electrode structure is coated with an enzyme-polymer layer, for example.

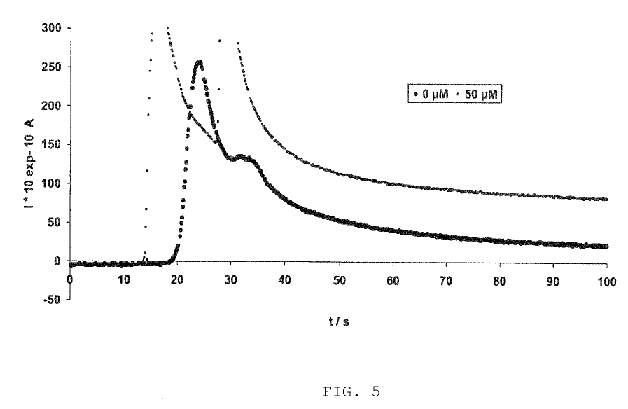

FIG. 4 shows schematically a holder with a Peltier element (4.1), a contact block for a biosensor structure (4.3), a heat conducting bridge (4.2) and insertion grooves (4.4) for the sensor element.

FIG. 5 shows the electric current signal as a function of the lactate concentration in the respiratory condensate in an exemplary embodiment.

1. A method for collecting respiratory condensates and immediately determining their constituents, characterized in that the exhaled air is condensed on at least one functionalized or activated surface with cooling; the condensate droplets thereby formed are sent directly to an electrochemical sensor and/or biosensor, the signals and/or reactions generated there are detected by means of a power supply system and a measurement, control and analysis system connected to the sensor and the constituents are determined, the condensate droplets reaching the sensor as a result of gravitational force, capillary forces and/or surface effects, depending on the geometric arrangement and dimensions of the surface, and substances that alter the conductivity of the condensate and/or substances that react chemically with the substances to be analyzed are used as the functionalizing or activating substances.

2. The method according to claim 1, wherein the condensation surface comprises a polymer layer which is functionalized or activated with substances that enter into an interaction and/or a chemical reaction with the condensate or with constituents that are to be analyzed and are contained therein.

3. The method according to claim 1, wherein the functionalized or activated surface comprises an organic or inorganic polymer layer of mixtures, preferably polyvinyl, polystyrene, polyacrylate, polyurethane, cellulose derivatives or silicon polymer compounds.

4. The method according to claim 1, wherein alkali salts or enzymes are used for functionalization.

5. The method according to claim 1, wherein the condensation surface is functionalized by covalent bonding of enzymes.

6. The method according to claim 1, wherein the amount of condensate is controlled as a function of time or the cooling temperature.

7. The method according to claim 1, wherein multiple condensation surfaces which are functionalized similarly or differently are used, and the condensate constituents are determined via cascades or by using similar or different sensors as a function of the substances to be determined.

8. The method according to claim 7, wherein different substances are determined in parallel, the functionalized condensation surfaces and/or sensors being equipped differently.

9. The method according to claim 1, wherein the cooling temperature, the point in time and duration of condensate collection are controlled by measuring the ambient temperature.

10. A device for collecting respiratory condensates and for immediately determining their constituents, comprising
a base element in the form of carrier or a holder, comprising a sensor unit, at least one Peltier element for cooling and a connecting heat conducting bridge, whereby the sensor unit has at least one condensation surface which is functionalized or activated with substances that alter the conductivity of the condensate or substances that react chemically with the substances to be analyzed, and comprising at least one electrochemical sensor or biosensor, whereby the functionalized or activated condensation surface and sensor or sensors are connected in such a way that the condensate can flow directly to the sensor through gravitational force, capillary forces or surface effects, depending on the geometric arrangement and dimensions of the condensation surface,
as well as a power supply system and an electronic measurement, control and analysis system.

11. The device according to claim 10, wherein the condensation surface is functionalized by covalent bonding of enzymes.

12. The device according to claim 11, wherein the condensation surface is coated with organic and/or inorganic polymers which are functionalized or activated with substances that enter into interactions or chemical reactions with the constituents in the respiratory air to be analyzed.

13. The device according to claim 10, wherein it comprises a temperature sensor that measures the ambient temperature.

14. The device according to claim 10, wherein several condensation surfaces which are connected to one or more Peltier elements via heat conducting bridges and the respective sensors are present, arranged so they are insulated from one another, the condensation surfaces having a similar or different functionalization and the sensors being similar and/or different.

15. The device according to claim 10, wherein the functionalized condensation surface is constructed based on polymers, ceramics, metals or a combination of these materials.

16. The device according to claim 10, wherein the condensation surface is coated with polymers, preferably polyvinyl polymers, polystyrenes, polyacrylates, polyurethanes, cellulose derivatives and/or silicon polymer compounds and/or polymer blends which are functionalized with alkali salts and/or enzymes.

17. The device according to claim 16, wherein the polymer coating is functionalized with alkali chlorides.

18. The device according to claim 17, wherein the polymer coating is functionalized with enzymes.

19. The device according to claim 17, wherein the polymer coating is functionalized with inorganic salts.

20. The device according to claim 10, wherein functionalization of the condensation surface is achieved by covalent bonding of enzymes to the condensation surface.

21. The device according to any claim 10, wherein the surface carrying the sensor is made of ceramic or a plastic, preferably a thermoplastic.

22. The device according to claim 10, wherein the respective condensation surface has defined geometric dimensions.

23. The device according to claim 10, wherein the sensor has a cover of a type such that it forms a space through which the condensate flows to the sensor through capillary forces.

24. The device according to claim 10, wherein the base element with the sensor unit, Peltier element and heat conducting bridge is provided in a housing equipped with slots or a grating.

25. The device according to claim 10, wherein it includes a headset and is designed so that the condensation surface can be positioned directly in front of the mouth of a test subject.

26. The device according to claim 10, wherein it is connected to a portable power supply system and analysis system.

27. Use of functionalized or activated condensation surfaces for deposition of condensate from the exhaled air for immediate and direct determination of the constituents thereof by means of essentially known electrochemical analysis equipment.
